SEQUENCE LISTING (2 sequences)

SEQ ID NO: 1 - protein, 4 aa, Artificial Sequence
  Synthetic Peptide Acetylation on Nitrogen
SDKP

SEQ ID NO: 2 - protein, 4 aa, Artificial Sequence
  Between the second and third residues, the peptide bond is replaced with the phosphonic bond (PO2CH2).
DFAA

(19) United States
(12) Patent Application Publication
(10) Pub. No.: US 2011/0104175 A1
(43) Pub. Date: May 5, 2011

(54) METHODS OF TREATMENT FOR PULMONARY FIBROSIS

(75) Inventors: Kenneth E. Bernstein, Los Angeles, CA (US); Sebastien Fuchs, Los Angeles, CA (US)

(73) Assignee: CEDARS-SINAI MEDICAL CENTER, Los Angeles, CA (US)

(21) Appl. No.: 12/995,401

(22) PCT Filed: Jun. 3, 2009
(86) PCT No.: PCT/US09/46158

§ 371 Date: Nov. 30, 2010

Related U.S. Application Data
(60) Provisional application No. 61/058,298, filed on Jun. 3, 2008.

(51) Int. Cl. A61K 39/395; A61K 38/07; A61P 11/00

(52) U.S. Cl. 424/146.1; 514/21.9

(57) ABSTRACT

The invention relates to compositions and methods for treating fibrosing lung diseases, reducing pulmonary cytokine production in animals, treating bleomycin induced lung disease and treating cancer. Specifically, the present subject matter incorporates inactivation of the N-terminal site of an angiotensin-converting enzyme and/or administration of AcSDKP.

### GOVERNMENT RIGHTS

This invention was made with U.S. Government support under NIH Grant No. K99 HL088000, NIH Grant No. R01 DK039777, and NIH Grant No. R01 DK051445. Thus, the U.S. Government may have certain rights in the subject matter hereof.

### FIELD OF THE INVENTION

The present field of the subject matter relates to methods for increasing tolerance to bleomycin and methods of treatment for pulmonary fibrosis; specifically to methods for treatment, tolerance, reduction and/or elimination of bleomycin induced fibrosis.

### BACKGROUND OF THE INVENTION

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Bleomycin is a glycopeptide antibiotic produced by the bacterium Streptomycin verticillus. When used as an anti-cancer agent, the chemotherapeutical forms are primarily bleomycin A₂ and B₂. Bleomycin has been effectively administered in the treatment of Hodgkin lymphoma (as a component of the ABVD regimen), squamous cell carcinomas, and testicular cancer, pleurodesis as well as plantar warts.

Bleomycin has potent anti-neoplastic properties for several neoplasms, particularly when used in conjunction with other cytostatic drugs (such as cisplatin and vinblastine). Bleomycin functions by binding to and damaging the DNA of tumor cells, thus terminating the cancerous cells. Some studies suggest that bleomycin also inhibits incorporation of thymidine into DNA strands. DNA cleavage by bleomycin depends on oxygen and metal ions, at least in vitro. It is believed that bleomycin chelates metal ions (primarily iron) producing a pseudoenzyme that reacts with oxygen to produce superoxide and hydroxide free radicals that cleave DNA. In addition, these complexes also mediate lipid peroxidation and oxidation of other cellular molecules.

However, drug administration of Bleomycin for cancer treatment is limited by a major side-effect, namely bleomycin-induced lung fibrosis. This side effect is due mostly to augmented concentration of reactive oxygen species, decrease in nicotinamide adenine dinucleotide (NAD) and adenosine triphosphate (ATP), and overproduction of mature collagen fibrils.

As bleomycin-induced lung fibrosis is easily reproduced in different species of mammals (e.g., mouse, rat, dog and pig), experimental models studying bleomycin injury in mice has been an effective and accurate means for investigating the cellular and molecular basis of lung fibrosis caused by bleomycin therapy.

Pulmonary fibrosis is a serious medical problem affecting more than 200,000 patients in the U.S. At present, there are few treatments available to counteract pulmonary fibrosis. Modulation of the renin-angiotensin system has been suggested, but the efficacy of such strategies remains uncertain and somewhat contradictory [Keogh, K. A., J. Standing, G. C. Kane, A. Terzic, and A. H. Limper Angiotensin II antagonism fails to ameliorate bleomycin-induced pulmonary fibrosis in mice. Eur. Respir. J. 25(4):708-714 (2005); Otsuka, M., H. Takahashi, M. Shiratori, H. Chiba, and S. Abe Reduction of bleomycin induced lung fibrosis by candesartan cilexetil, an angiotensin II type 1 receptor antagonist. Thorax 59(1) 31-38 (2004); Waseda, Y., M. Yasui, Y. Nishizawa, K. Inuzuka, H. Takato, Y. Ichikawa, A. Tagami, M. Fujimura, and S. Nakao Angiotensin II type 2 receptor antagonist reduces bleomycin-induced pulmonary fibrosis in mice. Respir. Res. 9:43 (2008); Ortiz, L. A., H. C. Champion, J. A. Lasky, F. Gambelli, E. Gozal, G. W. Hoyle, M. B. Beasley, A. L. Hyman, M. Friedman, and P. J. Kadowitz Enalapril protects mice from pulmonary hypertension by inhibiting TNF-mediated activation of NF-kappaB and AP-1. Am. J. Physiol. Lung Cell Mol. Physiol. 282(6): L1209-L1221 (2002)].

The renin-angiotensin system controls blood pressure and renal homeostasis in mammals. Angiotensin-converting enzyme (ACE) is the last enzyme of a cascade generating angiotensin II. The complete inactivation of ACE in mice results in a complex phenotype, including low blood pressure, anemia, male sterility and kidney malformations [Krege, J. H., S. W. John, L. L. Langenbach, J. B. Hodgin, J. R. Hagaman, E. S. Bachman, J. C. Jennette, D. A. O'Brien, and O. Smithies Male-female differences in fertility and blood pressure in ACE-deficient mice, Nature 375 (6527):146-148 (1995); Esther, C. R., Jr., T. E. Howard, E. M. Marino, J. M. Goddard, M. R. Capecchi, and K. E. Bernstein, Mice lacking angiotensin-converting enzyme have low blood pressure, renal pathology, and reduced male fertility. Lab Invest. 74(5):953-965 (1996)]. These mice, and data from several different experimental approaches, demonstrate physiologic roles for ACE beyond simple blood pressure control. For example, angiotensin II has been implicated in promoting fibrosis.

While a single polypeptide chain, functionally ACE is composed of two homologous but independent catalytic domains. These domains, termed the N- and C-domains, each bind a single Zn molecule which is required for catalysis [Wei, L., F. Alhenc-Gelas, P. Corvol, and E. Clauser. 1991. The two homologous domains of human angiotensin I-converting enzyme are both catalytically active, J. Biol. Chem. 266 (14):9002-9008 (1991)]. Whereas, both domains can cleave angiotensin I, and both domains can degrade bradykinin, the two domains of ACE have some important differences. For example, only the N-domain can use the tetrapeptide N-acetyl-seryl-aspartyl-lysyl-proline (AcSDKP; SEQ ID NO: 1), as a substrate, cleaving this peptide into inactive fragments [Rousseau, A., A. Michaud, M. T. Chauvet, M. Lenfant, and P. Corvol, The hemoregulatory peptide N-acetyl-Ser-Asp-Lys-Pro is a natural and specific substrate of the N-terminal active site of human angiotensin-converting enzyme. J. Biol. Chem. 270 (8):3656-366 (1995)]. Initially, AcSDKP was presented as a suppressor of bone marrow proliferation [Bonnet, D., R. Cesaire, F. Lemoine, M. Aoudjhane, A. Najman, and M. Guigon. The tetrapeptide AcSDKP, an inhibitor of the cell-cycle status for normal human hematopoietic progenitors, has no effect on leukemic cells, Exp. Hematol. 20(2):251-255 (1992)]. However, several in vivo studies has challenged this and suggested that AcSDKP may suppress fibrosis in models of tissue injury [Rasoul, S., O. A. Carretero, H. Peng, M. A. Cavasin, J. Zhuo, A. Sanchez-Mendoza, D. R. Brigstock, and N. E. Rhaleb Antifibrotic effect of Ac-SDKP and angiotensin-converting enzyme inhibition in hypertension, J. Hypertens. 22(3):593-603 (2004)]. ACE is the primary enzyme responsible for the degradation of AcSDKP; its actions in producing the pro-fibrotic molecule angiotensin II and in degrading the anti-fibrotic molecule AcSDKP suggests that ACE and the renin-angiotensin system may be instrumental in some fibrotic diseases.

Bleomycin induced lung injury is a well accepted model of lung fibrosis which has direct clinical correlates, since the anti-neoplastic use of bleomycin is limited by its lung toxicity; meta-analysis suggests that 10 to 50% of patients treated with bleomycin develop sufficient lung injury to necessitate cessation of treatment [Moore, B. B., and C. M. Hogaboam Murine models of pulmonary fibrosis, Am. J. Physiol. Lung Cell Mol. Physiol. 294(2):L152-L160 (2008)]. Of these, 1-3% die of lung injury [Chen, J., and J. Stubbe, Bleomycins: towards better therapeutics, Nat. Rev. Cancer 5(2):102-112 (2005)]. The present subject matter shows that N-KO mice are markedly resistant to the toxicity of bleomycin and that the accumulation of the ACE N-terminal peptide substrate AcSDKP, independent of angiotensin II, ameliorates bleomycin-induced lung fibrosis. The present subject matter provides a method for increasing tolerance to bleomycin as well as a method for treating other fibrosing lung diseases (e.g. bleomycin induced pulmonary fibrosis).

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1A shows images of the lung histology for wild type and N-KO mice, taken 14 days after bleomycin administration.

FIG. 1B is a graph depicting the fibrosis grade for wild type mice and N-KO mice.

FIG. 2 is a graph depicting the hydroxyproline content per dry lung for N-KO, C-KO and wild-type mice, taken two weeks after intratracheal injection of either saline or bleomycin.

FIG. 3 is a graph depicting the evolution of weight for mice before and after Bleomycin injection.

FIG. 4A is a graph depicting the hydroxyproline content per dry lung for N-KO, C-KO and wild-type mice, taken two weeks after intratracheal injection of either saline, bleomycin or the combination of intratracheal bleomycin and systemic S17092.

FIG. 4B is a graph depicting the cell count in the bronchoalveolar lavage from wild-type and N-KO mice treated with bleomycin in combination with S-17092 or Ac-SDKP.

FIG. 5 is a graph depicting the survival analysis of wild-type mice and N-KO mice treated with a high dose of intratracheal bleomycin.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which the subject matter belongs. Singleton et al., Dictionary of Microbiology and Molecular Biology 3rd ed., J. Wiley & Sons (New York, N.Y. 2002); March, Advanced Organic Chemistry Reactions, Mechanisms and Structure 4th ed., J. Wiley & Sons (New York, N.Y. 1992); and Sambrook and Russell, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present subject matter. Indeed, the present subject matter is in no way limited to the methods and materials described. For purposes of the present subject matter, the following terms are defined below.

“ACE” as used herein refers to Angiotensin-Converting Enzyme, an exopeptidase, which is a circulating enzyme that participates in the body's renin-angiotensin system. It is secreted by pulmonary and renal endothelial cells and catalyzes the conversion of decapeptide angiotensin I to octapeptide angiotensin II.

“AcSDKP” as used herein is the tetrapeptide acetyl Ser-Asp-Lys-Pro (SEQ ID NO: 1).

“Bleomycin” as used herein refers to a glycopeptide antibiotic produced by the bacterium Streptomyces verticillus.

“Cancer” and “cancerous” refer to or describe the physiological condition in mammals that is typically characterized by unregulated cell growth. Examples of cancer include, but are not limited to, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer.

“Mammal” as used herein refers to any member of the class Mammalia, including, without limitation, humans and nonhuman primates such as chimpanzees, and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus adult and newborn subjects, as well as fetuses, whether male or female, are intended to be including within the scope of this term.

“RXP407” as used herein refers to SEQ ID NO: 2, which is illustrated as follows:
Ac-Asp-_{(L)}Pheψ(PO₂—CH₂)_{(L)}Ala-Ala-NH₂

“Therapeutically effective amount” as used herein refers to that amount which is capable of achieving beneficial results in a mammal being treated. A therapeutically effective amount can be determined on an individual basis and can be based, at least in part, on consideration of the physiological characteristics of the mammal, the type of delivery system or therapeutic technique used and the time of administration relative to the progression of the disease, disorder or condition being treated.

“Treat,” “treating” and “treatment” as used herein refer to both therapeutic treatment and prophylactic or preventative measures, wherein the object is to prevent or slow down (lessen) the targeted pathologic condition, disease or disorder even if the treatment is ultimately unsuccessful. Those in need of treatment may include those already with the disorder as well as those prone to have the disorder or those in whom the disorder is to be prevented.

The present invention includes methods and compositions for treating fibrosing lung diseases, as well as methods and compositions for the treatment of other conditions, diseases and disorders where the alleviation of bleomycin-induced lung injury would be beneficial or desirable. The present invention is based, at least in part, on the finding that inactivating the N-terminal catalytic site of ACE leads to increased tolerance to bleomycin induced lung disease. While not wishing to be bound by any particular theory, it is believed that the accumulation of the tetrapeptide ACE substrate AcSDKP (SEQ ID NO: 1) is responsible for the aforementioned clinical effects.

In both humans and mice, pharmacologic inhibition of ACE increases plasma AcSDKP concentrations by 5-fold [Azizi, M., A. Rousseau, E. Ezan, T. T. Guyene, S. Michelet, J. M. Grognet, M. Lenfant, P. Corvol, and J. Menard Acute angiotensin-converting enzyme inhibition increases the plasma level of the natural stem cell regulator N-acetyl-seryl-aspartyl-lysyl-proline, J. Clin. Invest. 97(3):839-844 (1996)]. This is also true in ACE null and N-KO mice [Fuchs, S., H. D. Xiao, J. M. Cole, J. W. Adams, K. Frenzel, A. Michaud, H. Zhao, G. Keshelava, M. R. Capecchi, P. Corvol, and K. E. Bernstein, Role of the N-terminal catalytic domain of angiotensin-converting enzyme investigated by targeted inactivation in mice, J. Biol. Chem. 279(16):15946-15953 (2004)]. In contrast to angiotensin I or bradykinin, AcSDKP is a substrate of only the N-terminal ACE catalytic site, and it is not effectively cleaved by the C-terminal ACE catalytic site [Rousseau, A., A. Michaud, M. T. Chauvet, M. Lenfant, and P. Corvol, The hemoregulatory peptide N-acetyl-Ser-Asp-Lys-Pro is a natural and specific substrate of the N-terminal active site of human angiotensin-converting enzyme. J. Biol. Chem. 270 (8):3656-3661 (1995)]. Therefore, it is believed that inactivation of the N-terminal site of ACE reduces bleomycin-induced lung injury by increasing AcSDKP concentrations.

The present subject matter proves this deduction by using a mouse model, referred to here as N-KO, that was created by homologous recombination [Fuchs, S., H. D. Xiao, J. M. Cole, J. W. Adams, K. Frenzel, A. Michaud, H. Zhao, G. Keshelava, M. R. Capecchi, P. Corvol, and K. E. Bernstein, Role of the N-terminal catalytic domain of angiotensin-converting enzyme investigated by targeted inactivation in mice, J. Biol. Chem. 279 (16):15946-15953 (2004)). These mice were previously termed ACE 7/7 in the priority application. N-KO mice express a mutated form of ACE with an inactivation of the N-terminal catalytic domain. In this model, ACE is produced as a full length enzyme with only two point mutations which change ³⁹⁵His and ³⁹⁹His to lysines. In the absence of these histidines, the N-terminal domain does not bind zinc and is catalytically inactive. We also used mice called C-KO where it is the C-terminal catalytic site that was inactivated following a similar strategy [Fuchs, S., H. D. Xiao, C. Hubert, A. Michaud, D. J. Campbell, J. W. Adams, M. R. Capecchi, P. Corvol, and K. E. Bernstein, Angiotensin-converting enzyme C-terminal catalytic domain is the main site of angiotensin I cleavage in vivo, Hypertension 51(2):267-274 (2008)). In C-KO mice, ⁹⁹³His and ⁹⁹⁷His were mutated to lysine. In both lines of mice, ACE protein expression is under the normal native promoter, and therefore, there is a normal tissue and cellular distribution of the protein [Fuchs, S., H. D. Xiao, C. Hubert, A. Michaud, D. J. Campbell, J. W. Adams, M. R. Capecchi, P. Corvol, and K. E. Bernstein, Angiotensin-converting enzyme C-terminal catalytic domain is the main site of angiotensin I cleavage in vivo, Hypertension 51(2):267-274 (2008)].

It is worth emphasizing that the non-mutated catalytic sites of ACE in both N-KO and C-KO mice are fully active. Therefore, these mice are capable of converting angiotensin I to angiotensin II and, at steady state, have a normal blood pressure, normal kidney function and normal hematocrit parameters that are distinctly abnormal in ACE null mice [Krege, J. H., S. W. John, L. L. Langenbach, J. B. Hodgin, J. R. Hagaman, E. S. Bachman, J. C. Jennette, D. A. O'Brien, and O. Smithies, Male-female differences in fertility and blood pressure in ACE-deficient mice, Nature 375(6527):146-148 (1995); Esther, C. R., Jr., T. E. Howard, E. M. Marino, J. M. Goddard, M. R. Capecchi, and K. E. Bernstein, Mice lacking angiotensin-converting enzyme have low blood pressure, renal pathology, and reduced male fertility, Lab. Invest. 74(5):953-965 (1996)]. Thus, in contrast to virtually all other animal models, any differences noted between N-KO, C-KO and wild-type mice are almost certainly independent of the significant blood pressure reduction seen when using pharmacologic inhibitors of either ACE or angiotensin receptors.

The present subject matter demonstrates that the inactivation of the N-terminal site of ACE protects N-KO mice from bleomycin-induced lung injury and fibrosis. These mice develop significantly less pulmonary inflammation and collagen deposition, as compared to either C-KO or wild-type mice. Further, these mice show enhanced survival when exposed to a dose of bleomycin lethal to wild-type mice. As discussed above, these mice produce angiotensin II from the C-terminal of ACE. This is the physiologically dominant site of angiotensin II production by wild-type mice, and thus it is not surprising that the plasma concentration of angiotensin II in N-KO mice is identical to that of wild-type mice. Thus, the data strongly suggest that changes in angiotensin II do not explain the phenotype of the N-KO mice.

As mentioned above, AcSDKP is a specific substrate of the N-terminal site of ACE and may have antifibrotic properties. The major enzyme involved in the production of this tetrapeptide is prolyl-oligopeptidase which is efficiently inhibited by the Servier inhibitor S17092 [Cavasin, M. A., N. E. Rhaleb, X. P. Yang, and O. A. Carretero, Prolyl oligopeptidase is involved in release of the antifibrotic peptide Ac-SDKP, Hypertension 43(5):1140-1145 (2004)]. Administration of S17092 specifically reduces the concentration of AcSDKP, and not a panel of other peptides [Cavasin, M. A., N. E. Rhaleb, X. P. Yang, and O. A. Carretero, Prolyl oligopeptidase is involved in release of the antifibrotic peptide Ac-SDKP, Hypertension 43(5):1140-1145 (2004)]. To determine the importance of AcSDKP in the protection of N-KO mice to bleomycin, we treated these mice and wild-type mice with S17092 by daily intraperitoneal administration for two weeks following bleomycin administration. Accordingly, N-KO mice become equivalent to wild-type animals in their susceptibility to inflammation and fibrosis. Accordingly, the results further clarify the pivotal role of AcSDKP in curtailing bleomycin-induced lung injury and fibrosis.

The present subject matter further demonstrates the crucial contribution of the N-terminal catalytic domain of ACE in the development of bleomycin-induced pulmonary fibrosis. On the other hand, the inactivation of the C-terminal catalytic domain of ACE shows no such effects and C-KO animals are as susceptible to bleomycin-induced lung injury as wild-type mice. The data, and the experimental results using the prolyl-oligopeptidase inhibitor S17092, provides that AcSDKP accumulation plays a central role in protecting the lung against bleomycin injury.

Published work by Zhuo and colleagues have demonstrated a cellular specific binding site for AcSDKP [Zhuo, J. L., O. A. Carretero, H. Peng, X. C. Li, D. Regoli, W. Neugebauer, and N. E. Rhaleb, Characterization and localization of Ac-SDKP receptor binding sites using 125l-labeled Hpp-Aca-SDKP in rat cardiac fibroblasts, Am. J. Physiol. Heart Circ. Physiol. 292(2):H984-H993 (2007)]. One may hypothesize that the effects of AcSDKP are directly due to intracellular events triggered by specific binding of the peptide. While the intracellular actions of AcSDKP are not well characterized, some studies have suggested an effect on the TGF-β signaling pathway [Kanasaki, K., D. Koya, T. Sugimoto, M. Isono, A. Kashiwagi, and M. Haneda, N-Acetyl-seryl-aspartyl-lysyl-proline inhibits TGF-beta-mediated plasminogen activator inhibitor-1 expression via inhibition of Smad pathway in human mesangial cells, J. Am. Soc. Nephrol. 14 (4):863-872 (2003)]. The present subject matter identification of ACE and AcSDKP as having a major effect on bleomycin-induced lung fibrosis opens a new avenue of investigation for a means of reducing the progression of pulmonary fibrosis in response to a variety of pathologic processes. As current methods known in the art have little to offer patients with idiopathic pulmonary fibrosis, the present subject matter offers viable solutions for modulation and treatment of multiple fibrosing diseases, bleomycin induced lung disease, and reducing pulmonary cytokine production.

Exemplary agents that may substantially inactivate or hinder the effects of the N-terminal site of ACE may be site specific ACE inhibitors. There are two general types of ACE inhibitors: 1) the inhibitors commonly used in clinical practice which inhibit both catalytic domains, and 2) specialty peptides—used up to this point only in research—which specifically inhibit either N- or C-terminal ACE activity, but not both. For example, RXP 407, a peptide in which a phosphinic acid bond is used in place of a peptide bond, has a dissociation constant three orders of magnitude lower for the ACE N-domain than for the C-terminal domain [Dive V. et al. RXP 407, a phosphinic peptide, is a potent inhibitor of angiotensin I converting enzyme able to differentiate between its two active sites. Proc. Natl. Acad. Sci. USA. 96:4330-5 (1999)]. This compound is reported as stable in vivo and when used in a mouse increases the plasma level of Ac-SDKP as much as 6-fold [Junot C. et al., RXP 407, a selective inhibitor of the N-domain of angiotensin I-converting enzyme, blocks in vivo the degradation of hemoregulatory peptide acetyl-Ser-Asp-Lys-Pro with no effect on angiotensin I hydrolysis. J. Pharmacol. Exp. Ther. 297:606-11 (2001)]. When combined with Ac-SDKP infusion, a 16-fold elevation of plasma Ac-SDKP was obtained. Another phosphinic peptide, RXPA 380 is reported as being a C-terminal specific inhibitor, with a dissociation constant more than 3 orders of magnitude lower for the ACE C-domain than the N-domain [Georgiadis D. et al. Roles of the two active sites of somatic angiotensin-converting enzyme in the cleavage of angiotensin I and bradykinin: insights from selective inhibitors. Circ. Res. 25; 93:148-54 (2003)]. Again, this compound appears stable and effective in mice. Finally, Dr. Edward Sturrock, University of Cape Town, has prepared and tested a different chemical class of C-terminal specific ACE inhibitors, termed ketomethylene inhibitors [Watermeyer J M, Kroger W L, O'Neill H G, Sewell B T, Sturrock E D. Probing the basis of domain-dependent inhibition using novel ketone inhibitors of Angiotensin-converting enzyme. Biochemistry 47:5942-50 (2008)]. Again, there is a 3-order of magnitude difference in dissociation constant, with very little effect on the N-terminal of ACE.

Additional agents that may substantially inactivate or hinder the effects of the N-terminal site of ACE may include monoclonal antobodies. Treatment for cancer involving the use of monoclonal antibodies that bind only to cancer cell-specific antigens and induce an immunological response against the target cancer cell have been shown to be effective. As monoclonal antobodies function in almost any substance, it is possible to create monoclonal antibodies that specifically bind and inactivate or hinder a given substance, here the N-terminal site of ACE. Such monoclonal antobodies could also be modified for delivery of a toxin, radioisotope, cytokine or other active conjugate.

In conclusion, the subject matter disclosed, demonstrates the crucial contribution of the N-terminal catalytic domain of ACE in the development of bleomycin-induced pulmonary fibrosis. It is only this domain of ACE that degrades AcSDKP. Experiments using 517092 and AcSDKP itself, propose that AcSDKP accumulation, in the absence of ACE N-terminal catalytic activity, protects the lung against bleomycin injury. Transforming growth factor β (TGF-β) pathways are believed important in the development of bleomycin-induced injury, and Smad3 null mice, deficient in TGF-β signaling, are resistant to bleomycin injury. Interestingly, AcSDKP is able to inhibit TGF-13 pathway in some situations. In rats, AcSDKP prevented and even reversed collagen deposition in a model of heart infarction.

In addition the subject matter disclosed indicates that ACE inhibition, particularly of the N-terminal domain, alleviates some of the lung toxicity of bleomycin. Thus allowing an increased dose of the drug, increased length of treatment and perhaps application of bleomycin to additional types of tumors. The subject matter also postulates the novel method of manipulating ACE in combating other fibrosing diseases of the lung.

Therefore, one embodiment of the present invention provides for a composition for treating bleomycin-induced lung injury in a mammal, comprising an agent that substantially inactivates or hinders the biological effects of the N-terminal site of ACE. In an embodiment, the agent is and/or comprises AcSDKP (SEQ ID NO: 1), or an analog, derivative or equivalent thereof. In another embodiment, the agent is and/or comprises RXP 407 (SEQ ID NO: 2). In yet another embodiment, the agent is and/or comprises a monoclonal antibody. In another embodiment, the mammal is a human.

Another embodiment of the present invention provides for a composition for treating a fibrosing lung disease in a mammal, comprising an agent that substantially inactivates or hinders the biological effects of the N-terminal site of ACE. In an embodiment, the agent is and/or comprises AcSDKP (SEQ ID NO: 1), or an analog, derivative or equivalent thereof. In another embodiment, the agent is and/or comprises RXP 407 (SEQ ID NO: 2). In yet another embodiment, the agent is and/or comprises a monoclonal antibody. In another embodiment, the mammal is a human.

Another embodiment of the present invention is a method for treating bleomycin-induced lung injury in a mammal. The method comprises providing a composition comprising an agent that substantially inactivates or hinders the biological effects of the N-terminal site of ACE as described above, and administering a therapeutically effective amount of the composition to the mammal.

Another embodiment of the present invention is a method for treating a fibrosing lung disease in a mammal. The method comprises providing a composition comprising an agent that substantially inactivates or hinders the biological effects of the N-terminal site of ACE as described above, and administering a therapeutically effective amount of the composition to the mammal.

Yet another embodiment of the present invention is a method for treating cancer in a mammal. The method comprises treating the cancer with an anticancer agent that has deleterious effects on the mammal's lungs, and administering a therapeutically effective amount of an agent to the mammal that substantially inactivates or hinders the biological effects of the N-terminal site of ACE as described above, thereby reducing or eliminating the lung injury associated with the administration of the anticancer agent. In an embodiment, the anticancer agent is bleomycin or an equivalent, analog or derivative thereof.

In various embodiments, the present invention provides pharmaceutical compositions including a pharmaceutically acceptable excipient along with a therapeutically effective amount of the composition comprising an agent that substantially inactivates or hinders the effects of the N-terminal site of ACE as described above. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the present invention provides pharmaceutical compositions comprising an agent that substantially inactivates or hinders the effects of the N-terminal site of ACE. Exemplary agents may be ACE inhibitors. Specifically, N-terminus specific ACE inhibitors. In another embodiment, the exemplary agent is and/or comprises RXP 407 (SEQ ID NO: 2).

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical preparations may be made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

Preparation of Mice. We generated mice expressing site-inactivated ACE by homologous recombination. N-KO mice express a full length ACE protein where the N-terminal catalytic site was inactivated by mutating two zinc-binding histidines (³⁹⁵H and ³⁹⁹H) to lysines. In the C-KO strain, ⁹⁹³H and ⁹⁹⁷H, which are responsible for the zinc binding and catalytic activity of the C-terminal enzymatic site, were mutated to lysine. The generation of these two strains of mice has been described in detail prior. Both N-KO and C-KO mice are a genetic mix of strains 129 and C57BL/6. Age and gender-matched littermate controls were used in all studies. All mice used in this study have only one functional renin gene.
Drug Administration. The administration of bleomycin was performed under anesthesia by intratracheal instillation as previously described. A small skin incision was made to expose the trachea. A 25-gauge needle was inserted directly into the trachea without reaching the bifurcation and the animals were maintained on the surgical board with a 30 degree angle to ensure a correct diffusion of the drug or saline to both lungs. Bleomycin was solubilized in water, aliquoted, lyophilized and kept at −20 C. For each experiment a fresh aliquot was dissolved in saline and, unless otherwise indicated, administered in a dose of 1 mg/kg. The concentration of the solution was adapted to inject 2 μl/g, The skin was then sutured and the animal monitored until complete recovery.

To inhibit the release of AcSDKP from its precursor thymosin-4, we used the prolyl-oligopeptidase inhibitor 517092. This was a generous gift from the Institut de Recherche Servier, France. It was administrated once a day by intraperitoneal injection at a dose of 10 mg/kg from a freshly prepared solution. S17092 administration began the day before bleomycin instillation and continued until the animals were sacrificed.

Bronchoalveolar lavage and Hydroxyproline measurements. Two weeks after Neomycin was administered, mice were deeply anaesthetized by intraperitoneal administration of avertine. A PE50 tube was inserted and secured in the trachea through a neck incision. Bronchoalveolar lavage was performed by washing the whole lung with 1 ml of NaCl solution (9 g/l). This was repeated three times and a total of approximately 2.5 ml fluid was recovered. The entire lung was then carefully isolated, avoiding the extraparenchymal tissue. The lung was lyophilized overnight and the dried weight was recorded. The tissue was then hydrolyzed with 1 ml 6N HCl at 110° C. for 24 hours. 20 μl of the homogenate was added to 400 μl chloramine T solution (1.4% chloramine T, 10% isopropanol, 4% citric acid, 0.96% glacial acetic acid, 5.8% sodium acetate, 2.7% sodium hydroxide) and incubated at room temperature for 20 minutes. 400 μl Ehrlich's solution was added to each sample and the samples were further incubated 15 min at 65° C. Absorbance was measured at 550 nm. The concentration of hydroxyproline was then calculated by comparison to a standard curve generated for each experiment. Results are expressed as the average of duplicate measurements, in μg of hydroxyproline per mg of dry lung.
Survival analysis. Five mg of bleomycin per kg of body weight was injected to 15 wild-type and 15 N-KO mice. The time of death was reported and plotted on a survival diagram. The statistical analysis and significance were determined using Prism software to perform a Kaplan-Meier analysis.

### Example 1

A group of 8 N-KO and 8 wild-type control mice received 0.5 mg/kg body weight of bleomycin. These animals were sacrificed 14 days later. Under deep anesthesia, the thoracic cage was opened to expose the lung. A 25 G needle was secured in the trachea and 0.8 to 1 ml of 10% buffered formalin was injected until the lungs were inflated. The trachea was then ligatured. The whole lung was carefully collected in one block with the heart and immersed in formalin overnight. The tissue was then dehydrated and paraffin embedded using standard techniques. Five micron coronal sections were obtained from the central part of the lung and were stained with either Masson's trichrome or with hematoxylin and eosin. For each animal, both right and left lobes of the lung were examined by a pathologist blinded to the genotype of the mice.

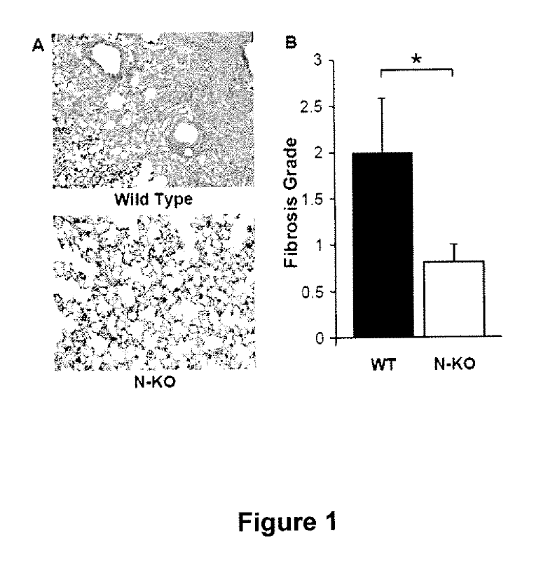

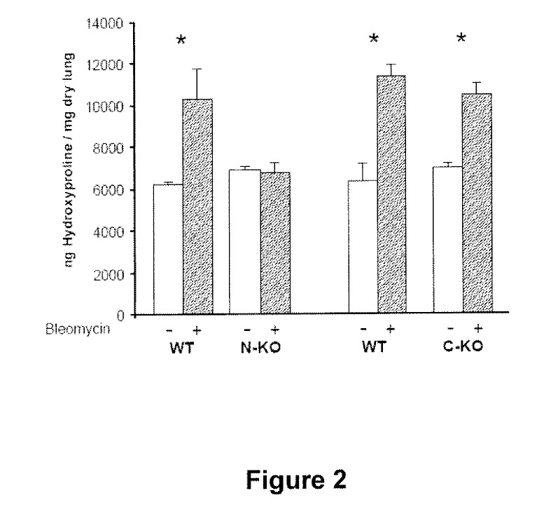

As expected, wild-type animals developed extensive lung injury, with pulmonary inflammation and deposition of collagen stripes. These features were much reduced in N-KO mice (FIGS. 1A and 1B). To objectively quantitate lung injury, we measured the amount of hydroxyproline in the whole lung. Before bleomycin administration, collagen content was similar in wild-type, N-KO and C-KO mouse lungs (FIG. 2), Two weeks after intratracheal bleomycin, wild-type and C-KO mice significantly increased the hydroxyproline content of their lungs. In contrast, N-KO mice did not increase pulmonary collagen levels. Thus, hydroxyproline levels confirm the preserved histology seen in N-KO mice.

### Example 2

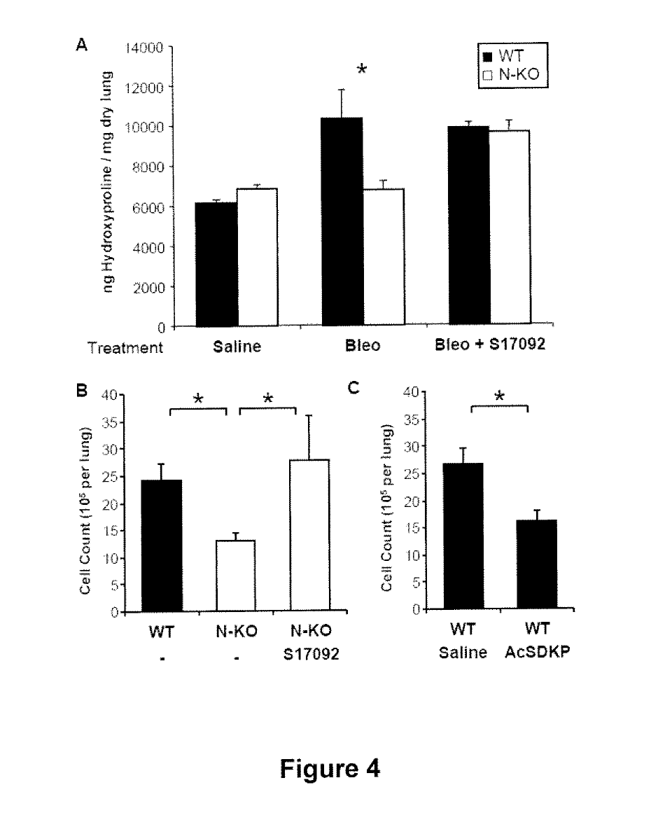

Previous study has determined that AcSDKP is substantially elevated in N-KO mice, which lack ACE N-terminal catalytic activity. This peptide is released from its precursor thymosin-β4 by the serine peptidase: prolyl oligopeptidase (POP). To determine the importance of AcSDKP in the mechanism of resistance to bleomycin injury, we inhibited the generation of this peptide with S17092, a POP inhibitor. This was accomplished by daily intraperitoneal injection of S17092 for two weeks following intratracheal instillation of bleomycin. Now, N-KO mice are susceptible to bleomycin induced fibrosis, as indicated by increased pulmonary hydroxyproline concentration (FIG. 4A). We also counted the number of cells present in a bronchoalveolar lavage performed two weeks after intratracheal bleomycin (FIG. 48). Without S17092, N-KO mice have a significant reduction in lavage cellularity compared to wild-type animals, which is consistent with the reduced inflammatory response in the N-KO lung (N-KO: 12.73±1.58 10⁵ cells per lung; WT: 24.21±3.10 10⁵ cells per lung). However, when S17092 is administrated to N-KO mice, the cell content of the bronchoalveolar lavage is not different from that of wild-type mice (N-KO: 27.71±8.14 10⁵ cells per lung).

### Example 3

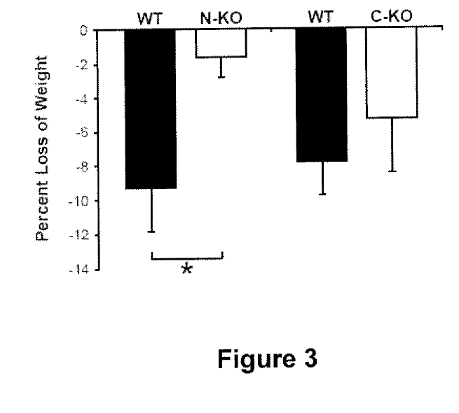

Weight loss is a typical feature secondary to bleomycin administration and is associated with the severity of the pulmonary lesions. Intratracheal instillation of bleomycin induced a weight loss of 9.26±2.52% in wild-type animals and only 1.60±1.23% in N-KO mice (FIG. 3). In contrast there was no significant difference in weight loss between C-KO mice and wild-type controls.

### Example 4

The data in the previous Examples suggests that the protection against bleomycin induced lung injury is substantially dependent on increased levels of AcSDKP present in N-KO mice. If this is so, then delivery of AcSDKP should have protective effects, even in wild-type mice. To test this, AcSDKP was administered to wild-type mice at a level of 1 mg/kg/day by osmotic minipump. On day 2, intratracheal bleomycin was administered, and two weeks later, mice were assessed by bronchoalveolar lavage. As seen in FIG. 4C, mice treated with AcSDKP presented with substantially reduced lavage cellularity, as compared to mice treated identically with a saline-filled minipump. However, in these wild-type mice with normal lung ACE activity, this protocol did not significantly reduce the collagen deposition, as assessed by hydroxyproline (data not shown).

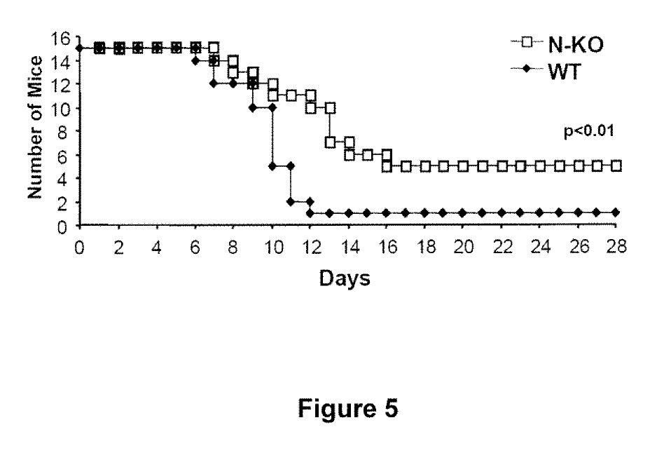

Intratracheal injection of our standard dose of bleomycin (1 mg/kg body weight) resulted in virtually no deaths of either wild-type or N-KO mice. However, a higher dose of bleomycin (5 mg/kg body weight) killed nearly all wild-type mice, with only 1 of 15 wild-type mice surviving 12 days after drug administration (FIG. 5). When challenged with the same dose of bleomycin, N-KO mice showed reduced mortality, with 11 or 15 mice surviving 12 days. Ultimately, 4 of 15 N-KO mice showed long-term survival (p<0.01 by Kaplan-Meyer analysis).

While particular embodiments of the present invention have been shown and described, it will be obvious to those skilled in the art that, based upon the teachings herein, changes and modifications may be made without departing from the subject matter and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1. A method of treating a fibrosing lung disease in a mammal, comprising:
providing a composition comprising an agent that substantially inactivates or hinders the biological effects of the N-terminal site of angiotensin-converting enzyme (ACE); and
administering a therapeutically effective amount of the composition to the mammal.

2. The method of claim 1, wherein the agent comprises AcSDKP (SEQ ID NO: 1) or an analog, derivative or equivalent thereof.

3. The method of claim 1, wherein the agent is AcSDKP (SEQ ID NO: 1).

4. The method of claim 1, wherein the agent is a N-terminal site inhibitor of ACE.

5. The method of claim 4, wherein the N-terminal site inhibitor of ACE is RXP 407 (SEQ ID NO: 2).

6. The method of claim 1, wherein the agent is a monoclonal antibody.

7. The method of claim 1, wherein administering further comprises using a route of administration selected from the group consisting of aerosol, nasal, oral, transmucosal, transdermal or parenteral.

8. The method of claim 1, wherein the mammal is a human.

9. A method of treating bleomycin-induced lung injury in a mammal, comprising:
providing a composition comprising an agent that substantially inactivates or hinders the biological effects of the N-terminal site of angiotensin-converting enzyme (ACE); and
administering a therapeutically effective amount of the composition to the mammal.

10. The method of claim 9, wherein the agent comprises AcSDKP (SEQ ID NO: 1) or an analog, derivative or equivalent thereof.

11. The method of claim 9, wherein the agent is AcSDKP (SEQ ID NO: 1).

12. The method of claim 9, wherein the agent is a N-terminal site inhibitor of ACE.

13. The method of claim 12, wherein the N-terminal site inhibitor of ACE is RXP 407 (SEQ ID NO: 2).

14. The method of claim 9, wherein the agent is a monoclonal antibody.

15. The method of claim 9, wherein administering further comprises using a route of administration selected from the group consisting of aerosol, nasal, oral, transmucosal, transdermal or parenteral.

16. The method of claim 9, wherein the mammal is a human.

17. A method of treating cancer in a mammal, comprising:
administering an anticancer agent to the mammal, the anticancer agent imparting deleterious effects on the mammal's lungs;
providing a composition comprising an agent that substantially inactivates or hinders the biological effects of the N-terminal site of angiotensin-converting enzyme (ACE); and
administering a therapeutically effective amount of the composition to the mammal.

18. The method of claim 17, wherein the agent comprises AcSDKP (SEQ ID NO: 1) or an analog, derivative or equivalent thereof.

19. The method of claim 17, wherein the agent is AcSDKP (SEQ ID NO: 1).

20. The method of claim 17, wherein the agent is a N-terminal site inhibitor of ACE.

21. The method of claim 20, wherein the N-terminal site inhibitor of ACE is RXP 407 (SEQ ID NO: 2).

22. The method of claim 17, wherein the agent is a monoclonal antibody.

23. The method of claim 17, wherein administering the composition further comprises using a route of administration selected from the group consisting of aerosol, nasal, oral, transmucosal, transdermal or parenteral.

24. The method of claim 17, wherein the mammal is a human.

25. A composition for the treatment of a fibrosing disease in a mammal, comprising:
an agent that substantially inactivates or hinders the biological effects of the N-terminal site of angiotensin-converting enzyme (ACE); and
a pharmaceutically acceptable vehicle and/or a pharmaceutically acceptable carrier.

26. A composition for the treatment of bleomycin-induced lung disease in a mammal, comprising:
an agent that substantially inactivates or hinders the biological effects of the N-terminal site of angiotensin-converting enzyme (ACE); and
a pharmaceutically acceptable vehicle and/or a pharmaceutically acceptable carrier.
